(19) United States
(12) Patent Application Publication
(10) Pub. No.: US 2019/0255224 A1
(43) Pub. Date: Aug. 22, 2019

(54) MEDICAL BALLOON, METHOD FOR MANUFACTURING MEDICAL BALLOON, AND BALLOON CATHETER

(71) Applicant: OLYMPUS CORPORATION, Tokyo (JP)

(72) Inventor: Yuri TAKEUCHI, Tokyo (JP)

(73) Assignee: OLYMPUS CORPORATION, Tokyo (JP)

(21) Appl. No.: 16/255,266

(22) Filed: Jan. 23, 2019

(30) Foreign Application Priority Data
Feb. 19, 2018 (JP) 2018-027125

(51) Int. Cl. A61L 29/06; A61M 25/10; B29C 49/00; B29C 49/04

(52) U.S. Cl. CPC A61L 29/06; A61M 25/1029; B29K 2077/00; B29C 49/04; A61M 2205/0216; B29C 49/0005

(57) ABSTRACT

A medical balloon includes: a polyamide elastomer; and a polyamide resin, wherein a mass ratio of the polyamide resin to the polyamide elastomer is 50/100 or more and 200/100 or less.

### BACKGROUND

### Technical Field

The present invention relates to a medical balloon, a method for manufacturing a medical balloon, and a balloon catheter.

Priority is claimed on Japanese Patent Application No. 2018-027125, filed Feb. 19, 2018, the content of which is incorporated herein by reference.

### Background Art

A medical balloon catheter (including a balloon dilator) is used for dilation of a stenosis in a living body lumen. For example, a balloon catheter expands a stenosis in the esophagus, ureter, bile duct, blood vessel, and the like.

For example, a balloon of a balloon catheter described in Japanese Unexamined Patent Publication, First Publication No. 2013-146505 is formed from a laminated film. In the laminated film, a polyamide elastomer layer is provided inside a polyamide layer. The molecular weight distribution (weight average molecular weight Mw/number average molecular weight Mn) of the whole balloon in the balloon catheter is 3 to 10.

Important properties of balloons of balloon catheters are compliance and pressure resistance. The compliance represents a diameter expansion amount per unit pressure (outer diameter change amount). The pressure resistance represents the pressure leading to the bursting of the balloon.

For dilating the stenosis, it is preferable that the compliance of the balloon is higher. However, if the compliance is high, the pressure resistance of the balloon tends to decrease.

In the technique described in Japanese Unexamined Patent Publication, First Publication No. 2013-146505, a pressure resistance performance and a passage performance are improved by suppressing the compliance to a low level. Specifically, the compliance of the balloon in Japanese Unexamined Patent Publication, First Publication No. 2013-146505 is 0.013 mm/atm or less.

### SUMMARY

A medical balloon includes: a polyamide elastomer; and a polyamide resin. A mass ratio of the polyamide resin to the polyamide elastomer is 50/100 or more and 200/100 or less.

The mass ratio of the polyamide resin to the polyamide elastomer may be 100/100 or more and 150/100 or less.

The polyamide elastomer may include at least one of a polyether block amide of a polyamide 11 series and a polyether block amide of a polyamide 12 series.

The polyamide resin may include at least one of polyamide 11 and polyamide 12.

At least one of the polyamide elastomer and the polyamide resin may be crosslinked.

A method for manufacturing a medical balloon includes: kneading at least a resin material containing a polyamide elastomer and a polyamide resin; forming a tubular parison from the kneaded resin material; and blow-molding the parison using a blow mold. In the resin material, a mass ratio of the polyamide resin to the polyamide elastomer is 50/100 or more and 200/100 or less. The blow mold has a molding surface for transferring a shape of the medical balloon to the parison.

The method for manufacturing the medical balloon may further include: crosslinking the resin material before or during molding of the parison.

When crosslinking the resin material, at least one of the polyamide elastomer and the polyamide resin may be crosslinked with one or more crosslinking agents selected from the group consisting of a carbodiimide, an acid anhydride, an isocyanate, and an oxazoline crosslinking agent.

The crosslinking agent may be a carbodiimide.

When the resin material is crosslinked, a temperature of the resin material may be 260° C. or more and 310° C. or less.

A balloon catheter includes the medical balloon described above.

### BRIEF DESCRIPTION OF THE DRAWINGS

FIG. 1 is a schematic front view showing an example of a balloon catheter according to an embodiment of the present invention.

FIG. 2 is a schematic cross-sectional view showing an example of a medical balloon according to an embodiment of the present invention.

FIG. 3 is a schematic cross-sectional view showing a metal mold used in a method of manufacturing a medical balloon according to an embodiment of the present invention.

### DETAILED DESCRIPTION OF THE PREFERRED EMBODIMENTS

Hereinafter, a medical balloon and a balloon catheter according to embodiments of the present invention will be described with reference to the accompanying drawings.

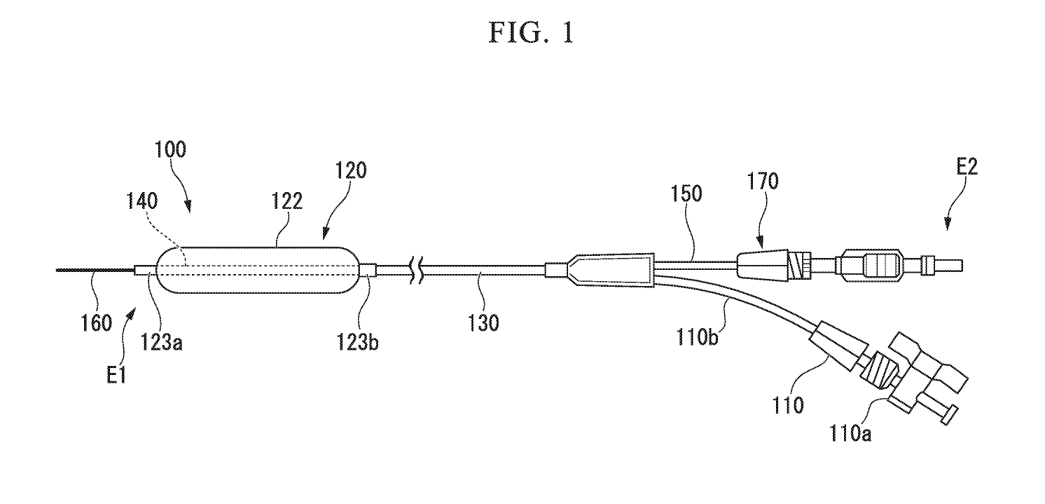

FIG. 1 is a schematic front view showing an example of a balloon catheter according to an embodiment of the present invention.

As shown in FIG. 1, a balloon catheter 100 of the present embodiment includes a hub 110, a proximal shaft 130, a balloon 120 (a medical balloon), and a guide wire lumen tube 150.

The shape of the balloon catheter 100 is an elongated shape as a whole. The balloon 120 is provided at the first end E1 (the end at the left side of the drawing) of the balloon catheter 100. The hub 110 and the guide wire lumen tube 150 are provided at the second end E2 (the end at the right side of the drawing) of the balloon catheter 100.

The balloon catheter 100 is operated by an operator. During use of the balloon catheter 100, the first end E1 is inserted into the body of a patient. During use of the balloon catheter 100, the second end E2 is kept outside the body of the patient. The operator operates the balloon catheter 100 near the second end E2.

In this specification, the tip of a member in a specific direction is referred to as an “end” unless otherwise noted. Although not limited to “end”, in a member, a portion closer to “end” in the specific direction is called “end portion”.

For example, in the direction from the second end E2 to the first end E1, the end of the first end E1 is called the “first end”. In the direction from the first end E1 to the second end E2, the end of the second end E2 is called the “second end”.

In the following, “distal side” and “proximal side” are used for indicating the relative position in the longitudinal direction of the balloon catheter 100. In the longitudinal direction of the balloon catheter 100, if position A is closer to the first end than position B, position A is more “distal” than position B.

In the longitudinal direction of the balloon catheter 100, when position A is closer to the second end than position B, position A is more “proximal” than position B.

In the members within the balloon catheter 100, the most distal end on each member is referred to as the “distal end”. In the members within the balloon catheter 100, the most proximal end of each member is referred to as “proximal end”.

The balloon catheter 100 is used, for example, for dilating and treating a stenosis of a lumen in a living body. When the stenosis is dilated by the balloon catheter 100, at least the balloon 120 is inserted into the stenosis. A fluid is supplied to the balloon 120. The fluid expands the diameter of the balloon 120.

The fluid may be liquid or gaseous. Examples of the fluid include a contrast medium, helium gas, physiological saline, carbon dioxide (CO₂) gas, oxygen (O₂) gas, nitrogen (N₂) gas, air, and the like.

The hub 110 passes the fluid as described above. Fluid is supplied to the hub 110 from a fluid supply device (not shown). The fluid is pressurized for enlarging the balloon 120 described later. The fluid is pressurized by the fluid supply device. The hub 110 is connectable to the fluid supply device. As the fluid supply device, for example, an inflator or the like may be used. The fluid supply device can adjust the pressure of the fluid.

At the proximal end of the hub 110, a stopcock 110a is provided. The fluid supply device can be connected to the hub 110 through the stopcock 110a. The stopcock 110a opens and closes the flow path of the fluid.

A tube 110b is connected to the distal end portion of the hub 110. A fluid can flow inside the tube 110b.

The proximal shaft 130 is connected to the distal end of tube 110b.

The proximal shaft 130 is an elongated member. The proximal shaft 130 can be inserted into the body of the patient. The proximal shaft 130 has flexibility. The proximal shaft 130 comprises a first lumen (not shown). The first lumen penetrates the proximal shaft 130 in the longitudinal direction. The first lumen communicates with the tube 110b. For this reason, fluid can flow inside the first lumen.

Even if the fluid is pressurized for expanding a diameter of a balloon 120, which will be described later, the proximal shaft 130 is rigid so as not to expand substantially.

On the distal side of the proximal shaft 130, a distal shaft 140 extends from the distal end face of the proximal shaft 130. The outer diameter of the distal shaft 140 is smaller than the outer diameter of the proximal shaft 130.

In the following, a portion extending from the distal end face of the proximal shaft 130 is referred to as an extending portion of the distal shaft 140. The length (extended length) of the extending portion is a length that can be covered by the balloon 120 described later.

The balloon 120 is joined to the distal end of the distal shaft 140 and the distal end of the proximal shaft 130.

The distal shaft 140 is a shaft-like member. The distal shaft 140 extends in the longitudinal direction of the proximal shaft 130. The distal shaft 140 may have a lumen as necessary. The distal shaft 140 in the present embodiment has a third lumen (not shown) as an example. The third lumen penetrates the distal shaft 140 in the longitudinal direction. A guide wire 160 described later can be inserted into the third lumen.

The guide wire 160 guides the balloon catheter 100 into the body of the patient. The guide wire 160 is made of a linear member having flexibility. The guide wire 160 is inserted into the body of the patient before the balloon catheter 100 is inserted into the body of the patient.

For example, in the first example of the distal shaft 140, the distal shaft 140 may extend from the distal end face of the proximal shaft 130.

In this case, the whole of the distal shaft 140 is an extending portion. The distal shaft 140 is integrated with the proximal shaft 130. Furthermore, the proximal shaft 130 has a second lumen (not shown) different from the first lumen. The second lumen penetrates the proximal shaft 130 in the longitudinal direction. In the second lumen, the guide wire 160 can be inserted. Further, the second lumen of the proximal shaft 130 and the third lumen of the distal shaft 140 communicate with each other so that the guide wire 160 can be inserted therethrough.

For example, the first lumen in the proximal shaft 130 may surround the outer periphery of the second lumen. In this case, the first lumen and the second lumen are arranged substantially coaxially (including coaxial case) (coaxial type).

For example, the first lumen and the second lumen in the proximal shaft 130 may be arranged not to surround each other. In this case, the first lumen and the second lumen are arranged in parallel (biaxial type).

For example, in the second example of the distal shaft 140, the distal shaft 140 may be a tubular member longer than the proximal shaft 130. However, the outer diameter of the distal shaft 140 is smaller than the inner diameter of the first lumen.

In this case, the distal shaft 140 is inserted into the first lumen of the proximal shaft 130. In the second example, inside the proximal shaft 130, the first lumen surrounds the third lumen (coaxial type).

The distal end of the distal shaft 140 extends (distally) from the distal end face of the proximal shaft 130.

In the second example, the distal shaft 140 and the proximal shaft 130 are fixed to each other at any position in the longitudinal direction. Therefore, the extended length of the distal shaft 140 is constant.

The balloon 120 is a thin-walled cylindrical member made of resin. The balloon 120 includes a first connecting portion 123a, a second connecting portion 123b, and a balloon body 122.

In FIG. 1, the shape of the balloon 120 is schematically drawn. However, the balloon 120 depicted in FIG. 1 is expanded by the fluid.

The first connecting portion 123a is cylindrical. The first connecting portion 123a has a constant outside diameter. The first connecting portion 123a is formed at the distal end portion of the balloon 120. The first connecting portion 123a covers the distal end portion of the distal shaft 140. The first connecting portion 123a is joined to the outer peripheral surface of the distal shaft 140. The bonding state of the first connecting portion 123a is liquid-tight.

The joining means of the first connecting portion 123a and the distal shaft 140 is not particularly limited as long as the first connecting portion 123a and the distal shaft 140 can be joined to each other in a liquid-tight manner. For example, the joining means between the first connecting portion 123a and the distal shaft 140 may be an adhesive. For example, the joining means between the first connecting portion 123a and the distal shaft 140 may be heat fusion bonding.

The second connecting portion 123b is tubular. The second connecting portion 123b has a constant outside diameter. The second connecting portion 123b is formed at the proximal end portion of the balloon 120. The second connecting portion 123b covers the distal end portion of the proximal shaft 130. The second connecting portion 123b is joined to the outer peripheral surface of the proximal shaft 130. The bonding state of the second connecting portion 123b is liquid-tight.

The joining means between the second connecting portion 123b and the proximal shaft 130 is not particularly limited as long as the second connecting portion 123b and the proximal shaft 130 can be joined to each other in a liquid-tight manner. As a joining means between the second connecting portion 123b and the proximal shaft 130, a joining means similar to the joining means between the first connecting portion 123a and the distal shaft 140 can be used, for example.

The balloon body 122 is formed at the intermediate portion in the longitudinal direction of the balloon 120.

The balloon body 122 is sandwiched between the first connecting portion 123a and the second connecting portion 123b.

The balloon body 122 surrounds the extended portion of the distal shaft 140.

The distal shaft 140 passes through the balloon 120 in the longitudinal direction of the balloon 120.

The inner peripheral surface of the balloon body 122 can be separated from the outer peripheral surface of the extended portion of the distal shaft 140.

Although not specifically shown, the first lumen of the proximal shaft 130 communicates with the space inside the balloon body 122. No opening except for the first lumen is formed inside the balloon body 122. Therefore, when fluid is introduced from the first lumen, the fluid is confined in the space inside the balloon body 122 and outside the distal shaft 140.

Although not specifically shown, when fluid is not introduced into the balloon body 122, the balloon body 122 is folded. When the balloon body 122 is folded, the balloon body 122 can be disposed to be wound around the outer peripheral surface of the distal shaft 140. When the balloon body 122 is wound around, the outer diameter of the balloon body 122 is substantially equal to the outer diameter of the proximal shaft 130. Therefore, in a state in which the balloon body 122 is folded, the portion where the balloon body 122 is provided has a columnar outer diameter substantially equal in diameter to the proximal shaft 130.

The balloon body 122 may have a crease for forming such a folded state.

The balloon body 122 is deployed by introducing fluid into the interior of the balloon body 122 through the first lumen.

In this specification, a state in which the balloon body 122 is deployed without elastic deformation is referred to as a “natural state” of the balloon body 122. A state in which at least a part of the balloon body 122 is expanded more than the natural state as a result of the elastic deformation of the balloon body 122 is referred to as an “expanded diameter state” of the balloon body 122.

The details of the balloon 120 will be described after the overall description of the balloon catheter 100.

The guide wire lumen tube 150 is a tubular member having a guide wire lumen as a lumen. The guide wire lumen penetrates the guide wire lumen tube 150 in the longitudinal direction. A guide wire 160 can be inserted through the guide wire lumen.

The distal end of the guide wire lumen tube 150 is connected to the proximal end of the proximal shaft 130. The guide wire lumen is in communication with the third lumen of the distal shaft 140.

For example, in the first example of the distal shaft 140, the guide wire lumen is in communication with the second lumen of the proximal shaft 130 and is in communication with the third lumen of the distal shaft 140.

For example, in a second example of the distal shaft 140, the guide wire lumen is in communication with the third lumen at the proximal end of the distal shaft 140.

A guide wire port 170 is formed at the proximal end of the guide wire lumen tube 150.

The guide wire port 170 is provided with an insertion port. The insertion port is an opening that allows the guide wire 160 inserted through the guide wire lumen tube 150 to extend toward the proximal side.

An example of the detailed configuration of the balloon 120 will be described.

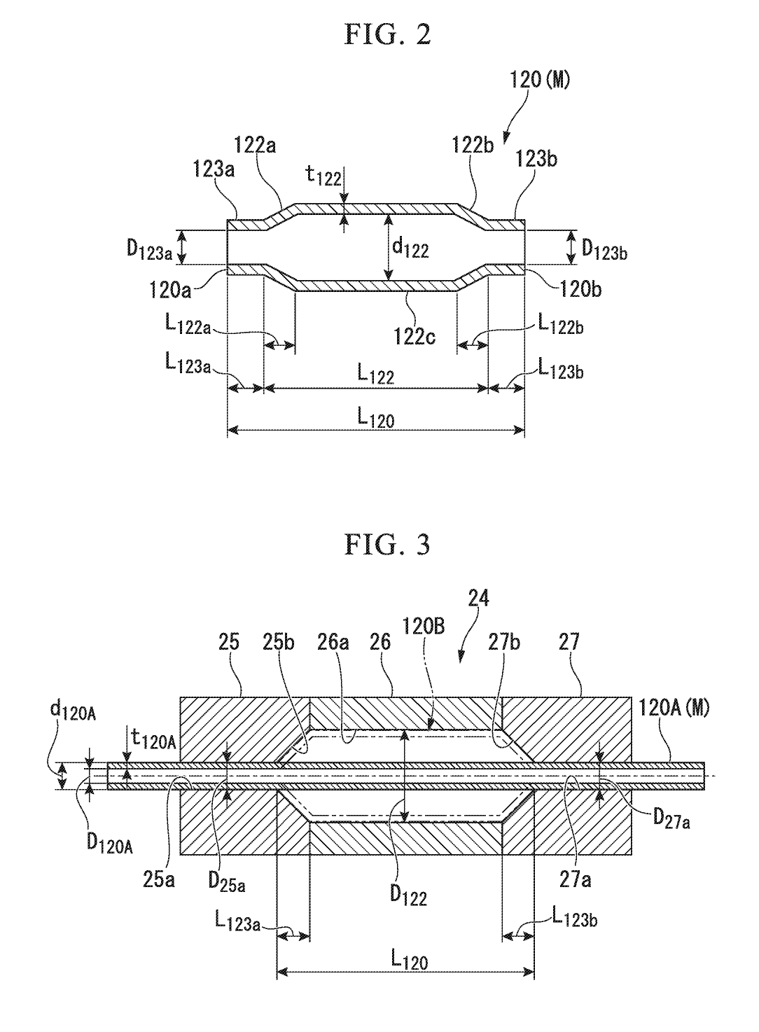

FIG. 2 is a schematic cross-sectional view showing an example of the medical balloon according to the embodiment of the present invention.

FIG. 2 shows a single balloon 120 of the present embodiment. For the balloon 120, “expanded state” and “natural state” are used to describe the situations above.

Below, the shape of the balloon 120 in the natural state is described. Even in the description of the balloon 120, “distal side” and “proximal side” are used similarly to the case of the assembled state of the balloon 120.

As shown in FIG. 2, the first connecting portion 123a has a cylindrical shape. The inner diameter of the inner peripheral surface of the first connecting portion 123a is D₁₂₃ₐ. The length of the first connecting portion 123a is L₁₂₃ₐ.

The inner diameter D₁₂₃ₐ is dimensioned such that the distal end portion of the distal shaft 140 can be inserted and can be joined to the outer peripheral surface of the distal shaft 140. For example, the inner diameter D₁₂₃ₐ may be substantially the same (including the same case) as the outer diameter of the distal end portion of the distal shaft 140.

The outer diameter of the distal shaft 140 also differs depending on the application of the balloon catheter 100. For example, the outer diameter of the distal shaft 140 may be 0.5 mm or more and 3.0 mm or less.

The length L₁₂₃ₐ is not particularly limited as long as necessary joining strength can be obtained for the first connecting portion 123a.

For example, the length L₁₂₃ₐ may be 5 mm or more and 30 mm or less.

The second connecting portion 123b has a cylindrical shape. The inner diameter of the inner peripheral surface of the second connecting portion 123b is D_{123b}. The length of the second connecting portion 123b is L_{123b}.

The inner diameter D_{123b} is dimensioned such that the distal end portion of the proximal shaft 130 can be inserted and can be joined to the outer peripheral surface of the proximal shaft 130. For example, the inner diameter D_{123b} may be approximately the same (including the same case) as the outer diameter of the distal end of proximal shaft 130.

The outer diameter of the proximal shaft 130 also varies depending on the application of the balloon catheter 100. For example, the outer diameter of the proximal shaft 130 may be 0.5 mm or more and 3.0 mm or less.

The length L_{123b} is not particularly limited as long as necessary bonding strength can be obtained for the second connecting portion 123b.

For example, the length L_{123b} may be 5 mm or more and 30 mm or less.

The balloon body 122 includes a curved surface portion 122a, a cylindrical portion 122c, and a curved surface portion 122b from the distal side toward the proximal side.

The curved surface portion 122a is connected to the proximal end of the first connecting portion 123a. The outer diameter of the curved surface portion 122a is smoothly enlarged from the distal side to the proximal side. The curved surface portion 122a tapers the outer shape of the distal end portion of the balloon body 122.

However, as long as the outer diameter of the curved surface portion 122a is smoothly enlarged from the distal side toward the proximal side, the rate of change of the gradient in the axial cross section of the curved surface portion 122a is not particularly limited. For example, the rate of change (change rate of outer diameter) of the gradient of the curved surface portion 122a may be constant.

In the example shown in FIG. 2, as an example, the change rate of the outer diameter of the curved surface portion 122a is constant. As a result, the curved surface portion 122a is, for example, a conical shape (linear tapered shape).

However, the change rate of the gradient of the curved surface portion 122a may change in the longitudinal direction of the balloon 120. For example, the curved surface portion 122a may have a curved surface shape bulging outward from the conical surface or a curved surface shape recessed inward from the conical surface. For example, the curved surface portion 122a may have a shape recessed inward from the cone on the distal side and may be changed to a shape bulging outward from the cone on the proximal side.

The maximum inner diameter of the curved surface portion 122a is d₁₂₂. The length of the curved surface portion 122a in the longitudinal direction of the balloon body 122 is L₁₂₂ₐ.

The cylindrical portion 122c is connected to the proximal end of the curved surface portion 122a. The inner diameter of the cylindrical portion 122c is d₁₂₂ as with the maximum inner diameter of the curved surface portion 122a.

The cylindrical portion 122c is provided for expanding the stenosis of the lumen in the living body. Examples of the lumen in a living body include an esophagus, a ureter, a bile duct, a blood vessel, and the like.

The length of the cylindrical portion 122c is longer than the length of the narrowed portion that is expanded by the balloon catheter 100. For example, the length of the cylindrical portion 122c may be 20 mm or more and 230 mm or less.

The outer diameter of the cylindrical portion 122c is expandable to the expanded diameter of the narrowed portion that is expanded by the balloon catheter 100. For example, the outer diameter of the cylindrical portion 122c may be 1 mm or more and 22 mm or less.

The curved surface portion 122b is connected to the proximal end of the cylindrical portion 122c and the distal end of the second connecting portion 123b. The curved surface portion 122b is tapered from the distal side to the proximal side. The curved surface portion 122b tapers the shape of the proximal end portion of the balloon body 122.

However, as long as the diameter is smoothly reduced from the distal side toward the proximal side at the curved surface portion 122b, the rate of change in the diameter is not particularly limited. For example, the change rate of the diameter of the curved surface portion 122b may be constant. For example, the change rate of the diameter of the curved surface portion 122b may change in the longitudinal direction of the balloon 120.

In the example shown in FIG. 2, as an example, the change rate of the diameter of the curved surface portion 122b is constant. Accordingly, the curved surface portion 122b is, for example, a conical shape.

The maximum inner diameter of the curved surface portion 122b is d₁₂₂. The length of the curved surface portion 122b in the longitudinal direction of the balloon body 122 is L_{122b}.

The shape of the curved surface portion 122b may be the same shape as that of the curved surface portion 122a except that the direction of diameter reduction is different. However, the shape of the curved surface portion 122b may be different from the shape of the curved surface portion 122a, in addition to the difference in the diameter reduction direction.

The length (effective portion length) of the cylindrical portion 122c of the balloon body 122 is L₁₂₂−(L₂₂ₐ+L_{122b}).

The thickness of each part of the balloon 120 may be equal to each other. However, the thickness of each part in the balloon 120 may not be equal to each other. In particular, the thickness of each part of the balloon 120 may vary, for example, due to molding reasons. The thickness of each part of the balloon 120 may be changed, for example, for controlling the shape at the time of diameter expansion.

The thickness of the balloon body 122 in the balloon 120 is t₁₂₂.

For example, the thickness t₁₂₂ may be 20 μm or more and 80 μm or less.

The balloon 120 is formed of a resin material M containing a polyamide elastomer (TPA, ThermoPlastic polyAmid elastomer) and a polyamide resin. The resin material M is a polymer blend (alloy) in which at least a polyamide elastomer and a polyamide resin are mixed at a micro level. In the present embodiment, the balloon 120 has a single layer structure.

In the resin material M, the mass ratio of the polyamide resin to the polyamide elastomer is 50/100 (=0.5) or more and 200/100 (=2) or less. The mass ratio of the polyamide resin to the polyamide elastomer is more preferably 100/100 (=1) or more and 150/100 (=1.5) or less.

As described above, the balloon 120 is fixed to the distal shaft 140 and the proximal shaft 130 at the first connecting portion 123a and the second connecting portion 123b, respectively. When fluid is introduced from the first lumen of the proximal shaft 130, fluid pressure acts on the inner surface of the balloon body 122. The outer diameter of the balloon body 122 formed of the resin material M changes according to the pressure of the fluid.

The outer diameter change ratio of the balloon 120 per unit pressure of the fluid, that is, the diameter expansion amount, is called compliance. In the present embodiment, the compliance of the balloon 120 is defined by the diameter expansion amount of the outer diameter of the cylindrical portion 122c.

If the compliance is low, the pressure of the fluid to be applied increases to obtain the required expansion amount. That is, if the outer diameter of the balloon before expansion is the same, it means that the pressure required to expand the balloon 120 to the same diameter becomes higher as compared with the case of high compliance. Therefore, the balloon 120 is required to have higher pressure resistance. Further, in this case, since the time to pressurize the fluid according to the magnitude of the pressure also becomes relatively long, the time required for the expansion of the balloon necessary for the expanded treatment of the narrowed portion and the contraction of the balloon after the treatment also becomes relatively long.

On the other hand, if the compliance is high, the pressure of the fluid to be applied is low in order to obtain the required expansion amount. However, if the pressure of the fluid does not exceed the pressure required to expand the narrowed portion, the balloon 120 located in the narrowed portion does not expand in diameter. In this case, it is necessary to further pressurize the diameter of the narrowed portion. As a result, there is a possibility that the portion of the balloon body 122, which does not come into contact with the narrowed portion, is excessively expanded in diameter.

Further, in this case, since the change in a diameter expansion amount of the balloon body 122 corresponding to the change in pressure of the fluid becomes too large, there is a possibility that the operation by the operator becomes difficult.

The compliance of the balloon 120 may be, for example, 4.44 mm/MPa (0.45 mm/atm) or more and 8.88 mm/MPa (0.9 mm/atm) or less. The compliance of the balloon 120 is more preferably, for example, 6.91 mm/MPa (0.7 mm/atm) or more and 8.88 mm/MPa (0.9 mm/atm) or more. Here, 1 atm is 0.101325 MPa.

The pressure resistance strength of the balloon 120 is equal to or higher than the pressure of the fluid required to expand the diameter of the narrowed portion.

The pressure resistance strength of the balloon 120 is more preferable as it is higher than the pressure of the fluid required to expand the diameter of the narrowed portion.

The pressure resistance strength of the balloon 120 may be, for example, 1.06 MPa (10.5 atm) or more. The pressure resistance strength of the balloon 120 is more preferably, for example, 1.216 MPa (12 atm) or more.

The polyamide elastomer is a material that easily improves the compliance of the balloon 120. The polyamide resin is a material which easily improves the pressure resistance strength of the balloon 120. Therefore, by adjusting the mass ratio of the polyamide elastomer and the polyamide resin, the compliance and the pressure resistance strength of the balloon 120 are optimized.

As the polyamide elastomer, for example, polyether block amide may be used. Polyether block amide is one type of polyamide elastomer.

The polyether block amide has a block structure in which hard segments and soft segments are alternately arranged. The hard segment is polyamide. The soft segment is a polyether. Polyether block amide is excellent in moldability and flexibility.

As the hard segment polyamide, at least one of polyamide 11 series and polyamide 12 series is particularly preferably used. In this case, the water absorption of the balloon 120 is suppressed. Therefore, the dimensional stability and temporal stability of the balloon 120 are improved.

The Shore hardness of the polyether block amide is more preferably D65 or more and D80 or less.

Examples of the polyamide resin include polyamide 11, polyamide 12, polyamide 1010, polyamide 1012, semi-aromatic polyamide, and amorphous polyamide (PA PACM 12).

In particular, when at least one of polyamide 11 series and polyamide 12 series is used as the hard segment of the polyamide elastomer, it is more preferable that the polyamide resin contains at least one of polyamide 11 and polyamide 12. In this case, the compatibility between the polyamide elastomer and the polyamide resin is improved. When the resin material M is composed of such a polyamide elastomer and a polyamide resin, since the resin material M is a completely compatible system, the balloon 120 can be made transparent.

When the narrowed portion is expanded by the balloon catheter 100, it is sometimes observed, through the balloon 120, whether or not there is bleeding or the like using the endoscope. In this case, if the balloon 120 is transparent, observation by the endoscope becomes easy.

When amorphous polyamide is contained in the polyamide resin, the compliance and the pressure resistance strength of the balloon 120 can be improved compared with a single polyamide elastomer alone. However, since the amorphous polyamide has low compatibility with the polyamide elastomer, the pressure resistance is lower than in the case where at least one of polyamide 11 and polyamide 12 is contained in the polyamide resin. The same applies to the case where a semi-aromatic polyamide is used in place of the amorphous polyamide.

Furthermore, when amorphous polyamide, semi-aromatic polyamide, or the like is added, the balloon 120 tends to be colored unlike the case where at least one of polyamide 11 and polyamide 12 is added. For this reason, the affected part becomes difficult to see when observing with the endoscope.

The balloon 120 of this embodiment is oriented during molding. By controlling the orientation, the compliance and the pressure resistance of the balloon 120 are improved.

When the polymer material is oriented, the strength of the polymer material increases in the orientation direction. However, the polymer material hardly elongates in the orientation direction. Therefore, in the cylindrically shaped product oriented in the circumferential direction, the strength in the circumferential direction is improved. As a result, the pressure resistance of the molded product is improved. However, the molded product becomes difficult to expand. Therefore, by adjusting the degree of orientation in the circumferential direction, it is possible to adjust the pressure resistance and the expandability of the molded product.

The orientation of the resin can be realized by aligning the molecular chain direction of the resin at the time of molding. The direction of the molecular chain of the resin is aligned, for example, by stretching, pushing in, drawing in, or the like at the time of molding.

For example, when a resin in a flexible state is stretched in one direction under pressure, the direction of the molecules of the resin is aligned with the stretching direction. Therefore, the degree of orientation of the resin in the stretching direction is improved.

In the present embodiment, as will be described later, the parison is manufactured with the resin material M. The balloon 120 is manufactured by blow-molding the parison. The orientation state of the balloon 120 is controlled during blow molding.

It is more preferable that at least one of the polyamide elastomer and the polyamide resin in the resin material M is crosslinked in the balloon 120.

The resin material M may contain a crosslinking agent. The kind of the crosslinking agent is not particularly limited as long as at least one of the polyamide elastomer and the polyamide resin can be crosslinked. The crosslinking agent may crosslink end groups of the polyamide elastomer or polyamide resin.

For example, as the crosslinking agent, one or more crosslinking agents selected from the group consisting of a carbodiimide, an acid anhydride, an isocyanate, and an oxazoline crosslinking agent may be used.

As the crosslinking agent, a carbodiimide or an oxazoline crosslinking agent is more preferable because it is excellent in biocompatibility, reactivity, and stability over time.

In the crosslinking reaction, an acid anhydride and an isocyanate compound generate, for example, by-products such as water and low molecular weight compounds. By-products tend to degrade the polyamide elastomer. Since by-products inhibit the crosslinking reaction, the reaction efficiency decreases. When by-products are mixed with the resin material M, orientation crystallization at the time of blow molding described later is able to be inhibited. Therefore, the carbodiimide and the oxazoline crosslinking agent are more preferable to the acid anhydride and the isocyanate compound.

When the carbodiimide is used, it is more preferably a polymer type carbodiimide. A high molecular type carbodiimide can crosslink with an amino group, a carboxyl group, and a hydroxyl group. Therefore, the carbodiimide can react with a plurality of terminal functional groups in the polyamide elastomer and the polyamide resin. As a result, the carbodiimide reacts with the material to be crosslinked one by one while forming a branch.

In contrast, the oxazoline crosslinking agent can react with only the carboxyl group. Therefore, compared to the oxazoline crosslinking agent, the polymer type carbodiimide is superior in reaction efficiency. The polymer type carbodiimide can crosslink in a shorter time with a smaller addition amount.

Furthermore, the high molecular type carbodiimide also acts as a hydrolysis inhibitor. Therefore, the polymer type carbodiimide can prevent the deterioration of the polyamide elastomer in particular.

Further, the high molecular type carbodiimide does not generate a substance which inhibits the crosslinking reaction or a by-product which inhibits oriented crystallization at the time of blow molding. Therefore, by using the polymer type carbodiimide, it is possible to further improve compliance and pressure resistance.

When the resin material M in the balloon 120 is crosslinked, the molecular weight of the resin material M is increased. Therefore, the pressure resistance strength of the balloon 120 is improved. Further, since the crosslinked structure is formed, elasticity is improved, and good compliance is obtained.

However, if an excessive crosslinking reaction occurs, the formability such as extrusion molding or the like deteriorates.

For example, orientation is likely to be inhibited during blow molding. As a result, there is a possibility that the pressure resistance strength of the balloon 120 is lowered.

For preventing an excessive crosslinking reaction from taking place, the amount of the crosslinking agent added is more preferably equal to or less than the functional group equivalent.

According to the experiment conducted by the present inventors, the degree of orientation is improved when the molecular weight of the resin material M is increased by crosslinking as compared with the uncrosslinked resin material M as long as the stretching ratio is the same. It is more preferable that the resin material M is crosslinked also in that the orientation can be easily controlled in this way. In addition, when the molecular weight is increased moderately by crosslinking, the fluidity is stabilized, and thus the dimensional accuracy at the time of extrusion molding the crosslinked resin material M is higher than that at the time of extrusion molding the uncrosslinked resin material M.

The weight average molecular weight of the resin material M is more preferably 35,000 or more and 90,000 or less. The weight average molecular weight of the resin material M is more preferably 45,000 or more and 80,000 or less.

The degree of crosslinking is also expressed by the Melt Flow Rate (MFR) value of the resin material M. The MFR value of the resin material M may be 1.0 g/10 min or more and 8.0 g/10 min or less. The MFR value of the resin material M is more preferably 1.5 g/10 min or more and 6.0 g/10 min or less.

A method of manufacturing the medical balloon of the present embodiment will be described.

FIG. 3 is a schematic cross-sectional view showing a metal mold used in the method of manufacturing the medical balloon according to the embodiment of the present invention.

The method of manufacturing a medical balloon of the present embodiment includes a parison forming step and a blow molding step.

In the parison forming process, the parison 120A (see FIG. 3) is produced by the resin material M. The parison 120A is tubular.

The inner diameter and the thickness of the parison 120A are D_{120A} and t_{120A}, respectively. The inner diameter D_{120A} is equal to or smaller than the smaller one of D₁₂₃ₐ and D_{123b}.

The thickness t_{120A} is the thickness at which the above-mentioned t₁₂₂ is obtained at a diameter expansion rate described later. For example, t_{120A} may be 0.25 mm or more and 0.80 mm or less.

The outer diameter of the parison 120A is d_{120A}=D_{120A}+2×t_{120A}.

First, in order to manufacture the parison 120A, the resin material M is manufactured.

Each material contained in the resin material M is melt-kneaded in a kneader. The mass ratio of the polyamide resin to the polyamide elastomer in the resin material M is 50/100 or more and 200/100 or less. When the resin material M is crosslinked, the above-mentioned crosslinking agent is also added.

The molding method of the parison 120A is not particularly limited. For example, the parison 120A may be manufactured by extrusion.

Extrusion molding is carried out by a suitable extruder. To the extruder, a die for forming the cross-sectional shape of the parison 120A is attached.

For example, the resin material M is heated to, for example, an extrusion molding temperature in an extruder.

The extrusion molding temperature is a temperature at which the polymer melts in the resin material M. The extrusion molding temperature may be, for example, 180° C. or more and 300° C. or less. The extrusion molding temperature is more preferably from 200° C. to 280° C. inclusive.

The resin material M heated to the extrusion molding temperature melts. The resin material M is extruded from the die of the extruder.

The extruded parison 120A is cut to an appropriate length to form the balloon 120.

Once the predetermined parison 120A can be manufactured, the parison forming process is completed.

As described above, it is an example of a parison forming process to knead the resin material M with a kneader and then to manufacture the parison 120A with an extruder. In the case where the molding machine used for molding the parison 120A has a resin material kneading function, kneading of the resin material M and molding of the parison 120A may be performed by the same molding machine.

At least one of the polyamide elastomer and the polyamide resin may be crosslinked before the parison 120A is produced.

For example, when one or more kinds of crosslinking agents selected from the group consisting of a carbodiimide, an acid anhydride, an isocyanate, and an oxazoline crosslinking agent are used as the crosslinking agent, the crosslinking reaction proceeds due to heat during kneading of the resin material M. In this case, the resin material M at the time of kneading may be heated for keeping a temperature at which the crosslinking reaction is likely to proceed. The temperature of the resin material M during kneading may be, for example, 260° C. or more and 310° C. or less.

When the temperature of the resin material M is lower than 260° C., there is a possibility that the crosslinking reaction does not occur sufficiently.

When the temperature of the resin material M exceeds 310° C., thermal decomposition is likely to occur along with the crosslinking reaction, so that the molecular weight lowers. For this reason, oriented crystallization at the time of blow molding, which will be described later, is easily inhibited.

In order to promote the crosslinking reaction more reliably, the moisture content of the resin material M at the time of crosslinking may be 0.08% or less. The moisture content of the resin material M at the time of crosslinking is more preferably 0.02% or less.

Crosslinking may be carried out by solid phase polymerization in a high temperature vacuum environment. Furthermore, one or more crosslinking agents selected from the group consisting of a carbodiimide, an acid anhydride, an isocyanate, and an oxazoline crosslinking agents may be used.

In the blow molding process, as shown in FIG. 3, the molding die 24 is prepared. The parison 120A is blow-molded using the molding die 24.

The molding die 24 includes a first die 25, a second die 26, and a third die 27.

The first die 25 transfers the shapes of the outer peripheral surfaces of the first connecting portion 123a and the curved surface portion 122a to the parison 120A. The first die 25 has a cylindrical molding surface 25a (molding surface) and a tapered molding surface 25b (molding surface), respectively, corresponding to the outer peripheral surface of the first connecting portion 123a and the outer peripheral surface of the curved surface portion 122a. The inner diameter size D₂₅ₐ of the cylindrical molding surface 25a is equal to the outer diameter of the first connecting portion 123a. The inner diameter of the tapered molding surface 25b is expanded from D₂₅ₐ to D₁₂₂ within the range of the length L₁₂₃ₐ.

The second die 26 transfers the shape of the outer peripheral surface of the cylindrical portion 122c to the parison 120A. The second die 26 has a cylindrical molding surface 26a (forming surface) corresponding to the shape of the outer peripheral surface of the cylindrical portion 122c. The size of the inner diameter of the cylindrical molding surface 26a is equal to D₁₂₂.

The third die 27 transfers the shapes of the outer peripheral surfaces of the curved surface portion 122b and the second connecting portion 123b to the parison 120A. The third die 27 has a tapered molding surface 27b (molding surface) and a cylindrical molding surface 27a (molding surface), respectively, corresponding to the shapes of the outer peripheral surface of the curved surface portion 122b and the outer peripheral surface of the second connecting portion 123b. The inner diameter of the tapered molding surface 27b is reduced in diameter from D₁₂₂ to D₂₇ₐ within the range of the length L_{123b}. The size of the inner diameter of the cylindrical molding surface 27a is D₂₇ₐ. D₂₇ₐ is equal to the outer diameter of the second connecting portion 123b.

Although not shown, a heating means and a cooling means are arranged outside the molding die 24. As a heating means, a suitable heater is used. As the cooling means, a cooling pipe through which a low temperature fluid can flow is used.

As shown in FIG. 3, the parison 120A is disposed inside the molding die 24 through the cylindrical molding surfaces 25a, 27a of the molding die 24. In the parison 120A, the first end in the longitudinal direction is sealed. For the sealing of the first end portion, for example, a seal by heat melting, a seal by high frequency, a mechanical seal such as forceps, or the like may be used.

Further, to the second end portion opposite to the first end portion of the parison 120A, a pressurized gas supply portion (not shown) is connected. For example, the first end portion may be the end portion of the parison 120A on the side of the first die 25. For example, the second end portion may be the end portion of the parison 120A on the side of the third die 27.

The first end portion and the second end portion of the parison 120A are supported so as to be movable in the longitudinal direction by a support portion (not shown).

The parison 120A in the molding die 24 is heated by the heating means. The heating temperature is a temperature equal to or higher than the glass transition point of the resin material M and lower than the melting point. For example, the heating temperature may be, for example, 35° C. or more and 140° C. or less. For example, the heating temperature may be, for example, 40° C. or more and 80° C. or less.

Further, pressurized gas is supplied from the pressurized gas supply unit to the second end portion of the parison 120A. The pressure of the pressurized gas may be 1.0 MPa or more and 3.5 MPa or less. The pressure of the pressurized gas is more preferably 1.5 MPa or more and 3.0 MPa or less.

In this way, the parison 120A is heated and pressed in the molding die 24. The parison 120A is held in the molding die 24 for a certain period of time. For example, the parison 120A is held in the heated and pressurized state described above for 3 minutes.

Thereafter, the parison 120A is stretched in the direction of the right and left arrows in the drawing. The extending distance of the parison 120A in the horizontal direction in the drawing may be 10 mm or more and 100 mm or less, respectively. The stretching distances are more preferably 40 mm or more and 90 mm or less, respectively. The stretching distance is more preferably 50 mm or more and 80 mm or less, respectively.

In this way, as the parison 120A is stretched in the heated and pressurized state, the parison 120A in the molding die 24 expands under internal pressure. As a result, the parison 120A is brought into close contact with the inner wall surface of the molding die 24. In this manner, the molded body 120B (see the two-dot chain line in FIG. 3) including the balloon body 122 is blow-molded from the parison 120A.

In the cylindrical molding surface 26 a, the molded body 120B expands in the radial direction from the outer diameter of the parison 120A to the inner diameter of the cylindrical molding surface 26a. D₁₂₂/D_{120A} represents the diameter expansion ratio by blow molding.

Thereafter, the molded body 120B is annealed. For example, the processing time of the annealing treatment is 3 minutes. As a result, the shape of the inner wall surface of the molding die 24 is transferred to the molded body 120B.

After the annealing treatment, the cooling liquid is circulated in the cooling pipe, whereby the molding die 24 and the molded body 120B are cooled. The molded body 120B is cooled to room temperature (20° C.).

Thereafter, the pressurization of the molded body 120B is stopped. Thereafter, the molded body 120B is released from the molding die 24.

Both end portions in the longitudinal direction are cut with respect to the molded body 120B. As a result, the shapes of the first connecting portion 123a and the second connecting portion 123b are formed in the molded body 120B. The balloon 120 is manufactured from the molded body 120B. In this way, the blow molding process is completed.

The above blow molding step is an example.

For example, in the blow molding process, the annealing process may be omitted. In this case, when the inflation of the parison 120A is completed, cooling by the cooling means may be started immediately.

For example, in the blow molding step, the parison 120A may be cooled naturally without depending on the cooling means. Specifically, after the heating by the heating means is stopped, the molding die 24 may be cooled by natural heat radiation.

An extended portion of the distal shaft 140 and an end portion on the distal side of the proximal shaft 130 are inserted into the center portion of the balloon 120 manufactured in this manner. The first connecting portion 123a of the balloon 120 is joined to the distal end of the distal shaft 140. The second connecting portion 123b of the balloon 120 is joined to the distal end of the proximal shaft 130.

In this way, the balloon catheter 100 is manufactured.

In the balloon 120 produced in this way, the mass ratio of the polyamide resin to the polyamide elastomer is 50/100 or more and 200/100 or less, and thus that the balance between the compliance and the pressure resistance becomes favorable. Therefore, even if the compliance is high, the breakdown pressure is high.

According to the study result of the present inventor, the diameter expansion ratio has a high correlation with the degree of orientation in the circumferential direction of the balloon 120.

For example, when the diameter expansion ratio is 250% or more and 400% or less, an appropriate pressure resistance strength can be obtained depending on orientation. However, since the orientation is low, the balloon 120 is likely to expand. Therefore, a balloon 120 excellent in compliance can be obtained.

For example, when the diameter expansion ratio is 400% or more and 650% or less, appropriate compliance can be obtained since the orientation is not excessive. However, since the orientation is high, the pressure resistance strength of the balloon 120 is high. Therefore, the balloon 120 having excellent pressure resistance strength is obtained.

For example, when the diameter expansion ratio is 300% or more and 450% or less, a balloon 120 having good compliance and excellent pressure resistance strength can be obtained.

In order to dilate the stenosis in the body of the patient by means of the balloon catheter 100, the balloon 120 is first folded. Thereafter, the balloon catheter 100 is inserted into the constricted portion in the living body from the distal side.

In the present embodiment, the balloon catheter 100 is guided by the guide wire 160 and inserted. Therefore, the guide wire 160 is inserted through the guide wire lumen and the third lumen in the balloon catheter 100. The guide wire 160 extends distally from the third lumen of the distal shaft 140.

First, the distal end of the guide wire 160 extending from the distal shaft 140 is inserted into the body of the patient. Once the distal end of the guide wire 160 is inserted through the stenosis, the balloon catheter 100 is inserted into the body of the patient. The balloon catheter 100 follows the path of the guide wire 160 and is inserted into the body of patient.

When the balloon 120 is inserted through the narrowed portion, the fluid is supplied to the inside of the balloon 120. By pressurizing the fluid, the balloon 120 expands according to the pressure of the fluid. The narrowed portion is expanded by the pressing force due to the expansion of the balloon 120.

Once the stenosis has been expanded to the required size, the fluid in the balloon 120 is expelled proximally. As a result, the balloon 120 is folded. The balloon catheter 100 is guided by the guide wire 160 and removed from the body of the patient. Furthermore, the guide wire 160 is removed from the body of the patient.

This completes the expansion procedure using the balloon catheter 100.

As described above, in the balloon 120 of the present embodiment, even if the compliance is high, the withstand pressure can be improved. Further, if the compliance is the same, the balloon 120 can obtain a relatively high withstand pressure strength as compared with the conventional example.

When the balloon 120 is provided on the balloon catheter 100, due to the high compliance, it is possible to rapidly enlarge and reduce the diameter with a small applied pressure. Therefore, the operability of the expansion procedure is improved. Since the operation time of the dilation procedure is shortened, the dilation treatment can be performed promptly. At that time, since the balloon 120 has high pressure resistance strength, rupture can be prevented even if the applied pressure increases.

In the description of the above embodiment, the case where the balloon catheter 100 is guided by the guide wire 160 has been described. However, if the balloon 120 can be inserted into the narrowed portion, the guide wire 160 may not be used. In this case, the third lumen of the guide wire lumen tube 150 and the distal shaft 140 may be omitted.

In the above description of the embodiment, in the second example of the distal shaft 140, the proximal end of the distal shaft 140 and the distal end of the guide wire lumen tube 150 are connected to each other.

However, in the case of the second example, the distal shaft 140 may be replaced by the guide wire lumen tube 150. In this case, the guide wire lumen tube 150 is inserted through the first lumen of the proximal shaft 130. The guide wire lumen tube 150 extends from the proximal and distal end faces of the proximal shaft 130, respectively. The guide wire lumen tube 150 is fixed to the proximal shaft 130 at any position in the longitudinal direction.

In the above description of the embodiment, the case where the balloon 120 has the cylindrical portion 122c has been described. Therefore, the outer peripheral surface of the cylindrical portion 122c of the balloon 120 in the natural state and the expanded diameter state is a cylindrical surface. However, the outer peripheral surface of the medical balloon in the natural state and in the expanded diameter state may be formed in a non-cylindrical surface shape having different outer diameters in the longitudinal direction. For example, the outer circumferential surface of the medical balloon in the natural state and the expanded diameter state may be a tapered shape, a dogbone shape, or the like.

### EXAMPLES

Next, examples of the medical balloon of the above-described embodiment will be described together with comparative examples.

The compositions and evaluation results of the medical balloons of Examples 1 to 9 and Comparative Examples 1 and 2 are shown in Table 1 below.

TABLE 1

| | MEDICAL BALLOON | | | | | | | |
|---|---|---|---|---|---|---|---|---|
| | POLYAMIDE | | POLYAMIDE | | | DIAMETER | | |
| | ELASTOMER | | RESIN | | MASS | EXPANSION | CROSSLINKING | |
| | | BY MASS | | BY MASS | RATIO | RATIO | AGENT | |
| | MATERIAL | (A) | MATERIAL | (B) | B/A | (%) | MATERIAL | BY MASS |
| EXAMPLE 1 | TPA | 100 | PA12 | 50 | 0.5 | 420 | — | — |
| EXAMPLE 2 | TPA | 100 | PA12 | 100 | 1 | 420 | — | — |
| EXAMPLE 3 | TPA | 100 | PA12 | 100 | 1 | 360 | — | — |
| EXAMPLE 4 | TPA | 100 | PA12 | 150 | 1.5 | 420 | — | — |
| EXAMPLE 5 | TPA | 100 | PA12 | 150 | 1.5 | 360 | — | — |
| EXAMPLE 6 | TPA | 100 | PA12 | 200 | 2 | 360 | — | — |
| EXAMPLE 7 | TPA | 100 | PA PACM12 | 100 | 1 | 420 | — | — |
| EXAMPLE 8 | TPA | 100 | PA12 | 100 | 1 | 420 | CARBODIIMIDE | 3 |
| EXAMPLE 9 | TPA | 100 | PA12 | 100 | 1 | 360 | CARBODIIMIDE | 3 |
| COMPARATIVE | TPA | 100 | — | — | — | 420 | — | — |
| EXAMPLE 1 | | | | | | | | |
| COMPARATIVE | TPA | 100 | PA12 | 250 | 2.5 | 360 | — | — |
| EXAMPLE 2 | | | | | | | | |

| | EVALUATION RESULTS | | | | |
|---|---|---|---|---|---|
| | | | PRESSURE | | |
| | | | RESISTANT | | |
| | COMPLIANCE | | STRENGTH | | COMPREHENSIVE |
| | (mm/MPa) | JUDGMENT | (MPa) | JUDGMENT | EVALUATION |
| EXAMPLE 1 | 6.02 | ◯ | 1.14 | ◯ | ◯ |
| EXAMPLE 2 | 5.13 | ◯ | 1.22 | ⊚ | ⊚ |
| EXAMPLE 3 | 7.80 | ⊚ | 1.08 | ◯ | ⊚ |
| EXAMPLE 4 | 4.84 | ◯ | 1.24 | ⊚ | ⊚ |
| EXAMPLE 5 | 7.11 | ⊚ | 1.12 | ◯ | ⊚ |
| EXAMPLE 6 | 5.72 | ◯ | 1.14 | ◯ | ◯ |
| EXAMPLE 7 | 6.12 | ◯ | 1.12 | ◯ | ◯ |
| EXAMPLE 8 | 4.24 | ◯ | 1.32 | ⊚ | ⊚ |
| EXAMPLE 9 | 7.01 | ⊚ | 1.24 | ⊚ | ⊚ |
| COMPARATIVE | 6.71 | ◯ | 1.04 | X | X |
| EXAMPLE 1 | | | | | |
| COMPARATIVE | 4.15 | X | 1.16 | ◯ | X |
| EXAMPLE 2 | | | | | |

### Example 1

As shown in Table 1, the balloon 120 (“medical balloon” in Table 1) of Example 1 was manufactured from a resin material M having a composition of 100 parts by mass of a polyamide elastomer (“TPA” in Table 1) and 50 parts by mass of a polyamide resin. As the polyamide resin, polyamide 12 (“PA 12” in Table 1) was used.

The mass ratio (B/A) of the polyamide resin to the polyamide elastomer was 0.5. No crosslinking agent was contained in the resin material M.

After the resin material M was kneaded, the parison 120A of Example 1 was produced by extrusion molding. The outer diameter of the balloon after molding was 12 mm, and the dimensions of the metal mold and the parison inner diameter were adjusted so that the expansion ratio (D₁₂₂/D_{120A}) at the time of producing the balloon 120 from the parison 120A was 420%. The wall thickness and stretch amount of the parison were adjusted so that the balloon thickness was 50 μm.

The balloon 120 of Example 1 thus produced was joined to the distal shaft 140 and the proximal shaft 130. Heat welding was used as the joining means. In this way, the balloon catheter 100 of Example 1 was manufactured.

### Examples 2 to 7

The balloon 120 of Example 2 was manufactured in the same manner as in Example 1 except that 100 parts by mass of the polyamide resin in the resin material M was used. The mass ratio of Example 2 was 1.

The balloon 120 of Example 3 was manufactured in the same manner as in Example 2 except that the diameter expansion ratio was set to 360%. The inner diameter of the parison 120A was adjusted so that the diameter expansion ratio was 360%. The wall thickness and stretch amount of the parison were adjusted so that the balloon thickness was 50 μm.

The balloon 120 of Example 4 was manufactured in the same manner as in Example 1 except that the polyamide resin in the resin material M was 150 parts by mass. The mass ratio of Example 4 was 1.5.

The balloon 120 of Example 5 was manufactured in the same manner as in Example 4 except that the diameter expansion ratio was set to 360%. The inner diameter of the parison 120A of Example 5 was adjusted for increasing the diameter expansion ratio to 360%.

The balloon 120 of Example 6 was produced in the same manner as in Example 3 except that the polyamide resin in the resin material M was changed to 200 parts by mass. The mass ratio of Example 6 was 2.

The balloon 120 of Example 7 was manufactured in the same manner as in Example 2 except that amorphous polyamide (PA PACM 12) was used as the polyamide resin in the resin material M.

The balloons 120 of Examples 2 to 7 were used for manufacturing the balloon catheters 100 of Examples 2 to 7 as in Example 1.

The balloons 120 of Examples 1 to 7 are examples in the case where the resin material M does not contain a crosslinking agent.

### Examples 8 and 9

The balloon 120 of Example 8 was manufactured in the same manner as in Example 2 except that 3 parts by mass of the crosslinking agent was added. As the crosslinking agent, a polymer type carbodiimide was used.

The balloon 120 of Example 9 was manufactured in the same manner as in Example 3 except that 3 parts by mass of the crosslinking agent was added. As the crosslinking agent, the same high molecular type carbodiimide as in Example 8 was used. The resin material M containing a crosslinking agent used in the balloons 120 of Examples 8 and 9 had a weight average molecular weight of 42,000 and an MFR of 2.4 g/10 min.

The balloons 120 of Examples 8 and 9 were used for manufacturing the balloon catheters 100 of Examples 8 and 9 as in Example 1.

The balloons 120 of Examples 8 and 9 are examples in the case where the resin material M contains a crosslinking agent.

### Comparative Examples 1 and 2

The medical balloon of Comparative Example 1 was manufactured in the same manner as in Example 1 except that the polyamide resin was omitted. Therefore, the resin material of Comparative Example 1 was composed only of polyamide elastomer.

The medical balloon of Comparative Example 2 was manufactured in the same manner as in Example 3 except that the polyamide resin in the resin material was changed to 250 parts by mass.

Comparative Examples 1 and 2 were used for manufacturing balloon catheters of Comparative Examples 1 and 2 as in Example 1.

### (Evaluation)

For evaluation of Examples 1 to 9 and Comparative Examples 1 and 2, the compliance and the pressure resistance were measured.

In the compliance measurement, physiological saline was supplied as a pressurized fluid to the balloon catheter of each example and each comparative example. The balloon was expanded from its natural diameter of 12 mm to the point of rupture. When expanding the diameter, the relationship between pressure change and outer diameter was measured. The outer diameter was measured by a laser displacement measuring device. The compliance was calculated from the slope of the graph of the outer diameter against pressure.

In the evaluation of the compliance, “very good” (“⊚” in Table 1) is obtained when the compliance was 6.91 mm/MPa (0.7 mm/atm) or more and 8.88 mm/MPa (0.9 mm/atm) or less; “good” (“◯” in Table 1) is obtained when the compliance was 4.44 mm/MPa (0.45 mm/atm) or more and less than 6.91 mm/MPa (0.7 mm/atm); and “no good” (“x” in Table 1) is obtained when compliance was less than 4.44 mm/MPa (0.45 mm/atm).

In the pressure resistance strength measurement, physiological saline was supplied as a pressurized fluid to the balloon catheter of each example and each comparative example. The balloon was pressurized until it burst. The pressure resistance strength was obtained as the pressure of the fluid at the time of rupture.

In the evaluation of the pressure resistance strength, “very good” (“⊚” in Table 1) is obtained when the pressure resistance strength was 1.216 MPa (12 atm) or more; “good” (“◯” in Table 1) is obtained when the pressure resistance strength was less than 1.216 MPa (12 atm) and 1.064 MPa (10.5 atm) or more; and “no good” (“x” in Table 1) is obtained when the pressure resistance strength was less than 1.064 MPa (10.5 atm).

In the overall evaluation, “very good” (“⊚” in Table 1) is obtained when at least one of the evaluation of the compliance and the pressure resistance strength was “very good”; “good” (“◯” in Table 1) is obtained when both of the evaluation of the compliance and the pressure resistance strength were “good”; and “no good” (“x” in Table 1) is obtained when at least one of the compliance and the pressure resistance is “no good”.

### (Evaluation Results)

As shown in Table 1, the compliance of Examples 1 to 9 were 6.02 mm/MPa, 5.13 mm/MPa, 7.80 mm/MPa, 4.84 mm/MPa, 7.11 mm/MPa, 5.72 mm/MPa, 6.12 mm/MPa, 4.24 mm/MPa, and 7.01 mm/MPa, respectively.

Examples 3, 5, and 9 were evaluated as “very good”. Other examples were evaluated as “good”.

In contrast, the compliance of Comparative Examples 1 and 2 were 6.71 mm/MPa and 4.15 mm/MPa, respectively.

Comparative examples 1 and 2 were evaluated as “good” and “no good”, respectively. In Comparative Example 2, it is considered that the compliance was out of the allowable range due to the excessive mass ratio of the hard polyamide resin.

The pressure resistance strengths of Examples 1 to 9 were 1.14 MPa, 1.22 MPa, 1.08 MPa, 1.24 MPa, 1.12 MPa, 1.14 MPa, 1.12 MPa, 1.32 MPa, and 1.24 MPa, respectively.

Examples 3 to 5, 8 and 9 were evaluated as “very good”. Other examples were evaluated as “good”.

In contrast, the pressure resistance strengths of Comparative Examples 1 and 2 were 1.04 MPa and 1.16 MPa, respectively.

Comparative examples 1 and 2 were evaluated as “no good” and “good”, respectively. In Comparative Example 1, since it consists only of a soft polyamide elastomer, the pressure resistance is considered to be outside the allowable range.

From the above evaluation results, Examples 2 to 5, 8 and 9 were evaluated as “very good” as a comprehensive evaluation. Examples 1, 6, and 7 were evaluated as “good”.

In contrast, both of Comparative Examples 1 and 2 were evaluated as “no good”.

While the preferred embodiments of the present invention have been described in conjunction with the respective embodiments, the present invention is not limited to this embodiment and each example. Additions, omissions, substitutions, and other changes in the configuration are possible without departing from the spirit of the present invention.

Also, the invention is not limited by the foregoing description, but only by the scope of the appended claims.

What is claimed is:

1. A medical balloon comprising:
a polyamide elastomer; and
a polyamide resin,
wherein a mass ratio of the polyamide resin to the polyamide elastomer is 50/100 or more and 200/100 or less.

2. The medical balloon according to claim 1, wherein the mass ratio of the polyamide resin to the polyamide elastomer is 100/100 or more and 150/100 or less.

3. The medical balloon according to claim 1, wherein the polyamide elastomer includes at least one of a polyether block amide of a polyamide 11 series and a polyether block amide of a polyamide 12 series.

4. The medical balloon according to claim 1, wherein the polyamide resin includes at least one of polyamide 11 and polyamide 12.

5. The medical balloon according to claim 1, wherein at least one of the polyamide elastomer and the polyamide resin is crosslinked.

6. A method for manufacturing a medical balloon, the method comprising:
kneading a resin material containing at least a polyamide elastomer and a polyamide resin;
forming a tubular parison from the kneaded resin material; and
blow-molding the parison using a blow mold,
wherein, in the resin material, a mass ratio of the polyamide resin to the polyamide elastomer is 50/100 or more and 200/100 or less, and
the blow mold has a molding surface for transferring a shape of the medical balloon to the parison.

7. The method for manufacturing the medical balloon according to claim 6, further comprising:
crosslinking the resin material before or during the blow-molding of the parison.

8. The method for manufacturing the medical balloon according to claim 7, wherein,
when crosslinking the resin material, at least one of the polyamide elastomer and the polyamide resin is crosslinked with one or more crosslinking agents selected from the group consisting of a carbodiimide, an acid anhydride, an isocyanate, and an oxazoline crosslinking agent.

9. The method for manufacturing the medical balloon according to claim 8, wherein the crosslinking agent is a carbodiimide.

10. The method for manufacturing the medical balloon according to claim 7, wherein,
when the resin material is crosslinked, a temperature of the resin material is 260° C. or more and 310° C. or less.

11. A balloon catheter comprising the medical balloon according to claim 1.
